(19) United States
(12) Patent Application Publication
(10) Pub. No.: US 2006/0079951 A1
(43) Pub. Date: Apr. 13, 2006

(54) Guide catheter with attached stent delivery system

(75) Inventors: Mark J. Dolan, Santa Rosa, CA (US); Dwayne S. Yamasaki, Rohnert Park, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/962,316

(22) Filed: Oct. 8, 2004

(51) Int. Cl. A61F 2/06

(52) U.S. Cl. 623/1.11

(57) ABSTRACT

The invention provides a system and method for treating a vascular condition. The system comprises a guide catheter that includes a central lumen. A stent delivery device that includes a delivery catheter and a stent disposed on the delivery catheter is slidably received in the central lumen. A stop interface retains a portion of the delivery catheter within the central lumen while still allowing positioning of the stent beyond a distal end of the guide catheter. In use, the guide catheter with the retained stent delivery device is introduced into a vessel. The stent carried on the stent delivery device is expanded after being positioned at a location beyond a distal end of the guide catheter.

### TECHNICAL FIELD

This invention relates generally to biomedical systems for treating vascular conditions. More specifically, the invention relates to a guide catheter with an attached stent delivery system.

### BACKGROUND OF THE INVENTION

Stents are cylindrical-shaped devices that are radially expandable to hold open a segment of a vessel or other anatomical lumen after implantation into the lumen. Various types of stents are in use, including expandable and self-expanding stents. Expandable stents generally are conveyed to the area to be treated on balloon catheters or other expandable devices. For insertion, the stent is positioned in a compressed configuration along the delivery device, for example crimped onto a balloon that is folded or otherwise wrapped about a guidewire that is part of the delivery device. After the stent is positioned across a lesion, it is expanded by the delivery device, causing the diameter of the stent to expand. For a self-expanding stent, commonly a sheath is retracted, allowing expansion of the stent.

The stent acts as a scaffold to support the lumen in an open position. The increased interior vessel diameter facilitates improved blood flow. Configurations of stents include a cylindrical tube defined by a solid wall, a mesh, interconnected stents, or like segments. Exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau.

Catheters used to deliver stents are commonly about 120 centimeters long. While catheters this length are appropriate for delivering stents into vessels near the heart that have undergone percutaneous transluminal coronary angioplasty (PTCA), they can be inconveniently long and unnecessarily expensive for treating vessels such as the internal iliac, which is considerably nearer to a typical same-side femoral artery access site. Reduced blood flow through the internal iliac, can impair function of muscles, nerves, and organs. Delivering a stent into an internal iliac that has become occluded can open the vessel sufficiently to improve blood flow, thereby relieving the dysfunction.

Although the internal iliac is a short distance from a same-side femoral artery access site, guiding a catheter into the vessel can be difficult. Typically the catheter must negotiate a turn from the femoral artery into the internal iliac artery that is more acute than 90 degrees. This can require considerable effort and expertise on the part of the doctor performing the procedure when a typical PTCA catheter is used.

Therefore, it would be desirable to have a system and a method for treating a vascular condition that overcome the aforementioned and other disadvantages.

### SUMMARY OF THE INVENTION

One aspect of the present invention is a system for treating a vascular condition. The system comprises a guide catheter that includes a central lumen. A treatment device is disposed on a delivery catheter that is slidably received in the central lumen. A stop interface retains a portion of the delivery catheter within the central lumen of the guide catheter while still allowing positioning of the treatment device beyond a distal end of the guide catheter.

Another aspect of the present invention is a method for treating a vascular condition. A stent delivery device is retained within a guide catheter using a stop interface. The guide catheter with the retained stent delivery device is introduced into a vessel. A stent carried on the stent delivery device is positioned at a location beyond a distal end of the guide catheter. The stent is expanded.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a system for treating a vascular condition, in accordance with the present invention;

FIG. 2 is an illustration of the system of FIG. 1 being introduced into an internal iliac artery;

FIG. 3 is an illustration of a stop interface for the system of FIG. 1;

FIG. 4 is an illustration of another embodiment of a system for treating a vascular condition, in accordance with the present invention; and

FIG. 5 is a flow diagram of one embodiment of a method for treating a vascular condition, in accordance with the present invention.

### DETAILED DESCRIPTION OF THE PRESENTLY PREFERRED EMBODIMENTS

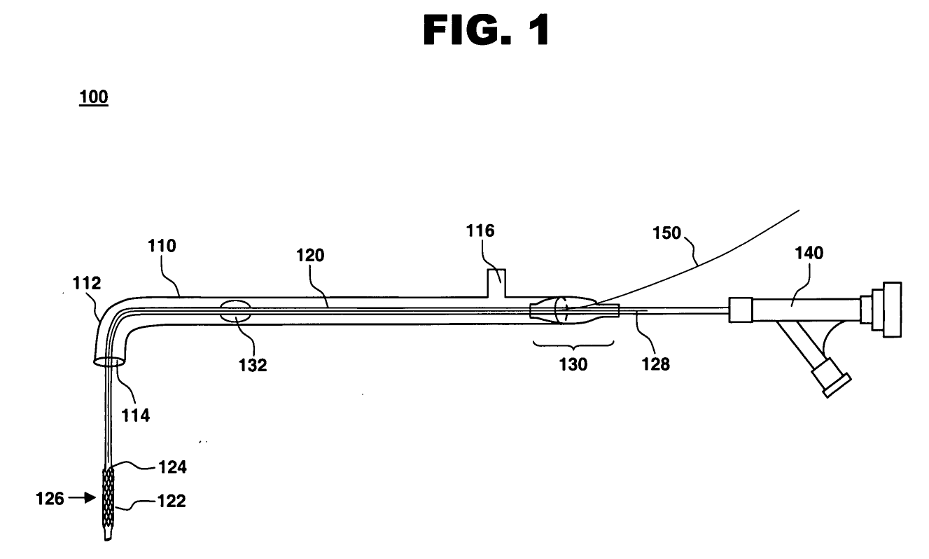

One aspect of the present invention is a system for treating a vascular condition. One embodiment of the system, in accordance with the present invention, is illustrated in FIG. 1 at 100. System 100 comprises a guide catheter 110, a delivery catheter 120, a treatment device 122, and a stop interface 130.

Guide catheter 110 may be any appropriate catheter known in the art, for example a catheter comprising stainless steel wire braided within a polymer. A distal portion of the guide catheter comprises a shaped, flexible tip 112 that is predisposed to bend at a predetermined angle. Tip 112 is flexible enough to assume a linear configuration when navigating a straight vessel but bends when a side branch of the vessel is encountered, thereby aiding in directing the tip into the side branch.

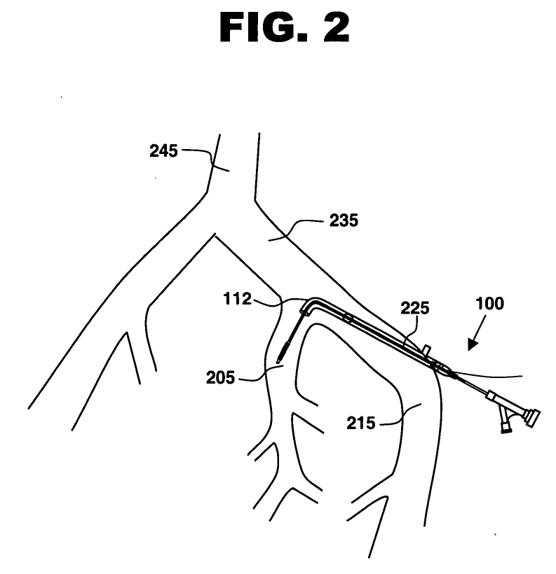

The tip may be customized for a particular use. For example, FIG. 2 shows system 100 with tip 112 angled to direct the system into an internal iliac artery 205 from a femoral artery 215, the system having been introduced through a percutaneous access site 225. The iliac and aorta are indicated at 235 and 245, respectively. For an application such as is shown in FIG. 2, the length of guide catheter 110 can be less than 50 centimeters.

Returning to FIG. 1, guide catheter 110 includes a central lumen 114 that extends through the catheter and receives delivery catheter 120. Central lumen 114 is sized to allow delivery catheter 120 to slide within guide catheter 110. Guide catheter 110 further includes a flush port 116 that is used, for example, for flushing the catheter prior to use or for introducing a bolus of contrast solution during use.

A treatment device, in the present embodiment stent 122, is disposed on delivery catheter 120, which may be any catheter known in the art that is appropriate for delivering the stent. Stent 122 may be made of a wide variety of medical implantable materials, including, but not limited to, stainless steel, nitinol, tantalum, ceramic, nickel, titanium, aluminum, polymeric materials, MP35N, platinum iridium, titanium ASTM F63-83 Grade 1, niobium, high carat gold K 19-22, and combinations thereof. In the embodiment shown in FIG. 1, stent 122 is an expandable stent, and delivery catheter 120 includes a balloon 124 that is used to expand the stent. In another embodiment, the stent may be eliminated and the balloon may act alone as a treatment device, for example when performing PTCA.

A therapeutic coating 126 may be disposed on at least a portion of the stent or balloon. The therapeutic coating may include, for example, an antineoplastic agent, an antiproliferative agent, an antibiotic, an antithrombogenic agent, an anticoagulant, an antiplatelet agent, an anti-inflammatory agent, combinations of the above, and the like.

Stop interface 130 prevents delivery catheter 120 from being accidentally disengaged from guide catheter 110. Stop interface 130 is positioned adjacent to the proximal end of guide catheter 110 and retains a portion of delivery catheter 120 within central lumen 114 of guide catheter 110. At the same time, stop interface 130 allows movement of delivery catheter 120 to permit stent 122 to be positioned beyond a distal end of guide catheter 110. As shown in FIG. 1, the proximal end of delivery catheter 120 extends outside of stop interface 130 and can be grasped to slide delivery catheter 120 into guide catheter 110 through stop interface 130, thereby positioning stent 122 beyond the distal end of guide catheter 110. Delivery catheter 120 may include a stop 132 to prevent the treatment device from being damaged by coming into contact with the stop interface. Stop 132 may be, for example, a droplet of adhesive deposited onto the outer surface of delivery catheter 120. An end user receives system 100 with delivery catheter 120 already attached to guide catheter 110.

In the present embodiment, stop interface 130 is sized and shaped to permit a portion of delivery catheter 120 to slide through the interface and into central lumen 114 of guide catheter 110. Stop interface 130 is also sized and shaped to allow a guidewire 150 to pass through the interface and into the lumen of delivery catheter 120. Delivery catheter 120 may include a longitudinally extending slit 128 that is opened by spreading means when delivery catheter 120 passes through stop interface 130, allowing the guidewire to be received within a lumen of the delivery catheter. Beyond the spreading means, the slit returns to a closed configuration under the influence of the inherent resiliency of the delivery catheter material.

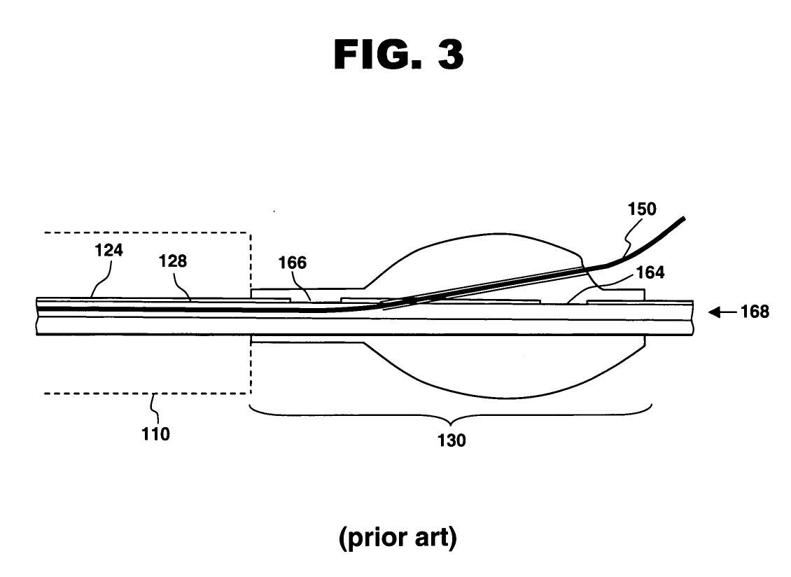

The described stop interface may function in a manner similar to that of catheter and guidewire exchange systems described in U.S. Pat. No. 4,988,356 to Crittenden et al. and U.S. Patent Application Publication No. 2003/0191491 to Duane et al. FIG. 3, in which like elements share like numbers with FIG. 1, illustrates one such system. As shown, the system includes proximal and distal spreading members 164 and 166 that allow guidewire 150 to be received within guidewire lumen 168 of delivery catheter 120.

In a variation of the present embodiment, the stop interface may instead be a hemostatic valve known in the art that is capable of retaining a portion of the delivery catheter within the central lumen of the guide catheter while still allowing positioning of the stent beyond the distal end of the guide catheter. A stop may be included to prevent the treatment device carried by the delivery catheter from coming in contact with the stop interface. In other variations in accordance with the present invention, the delivery catheter may be any over-the-wire catheter known in the art, or it may be directed through the guide catheter without using a guidewire.

A luer fitting 140 or other type of fitting may be attached to a proximal end of delivery catheter 120. The fitting may be in communication with a fluid source, thereby providing for inflation of balloon 124.

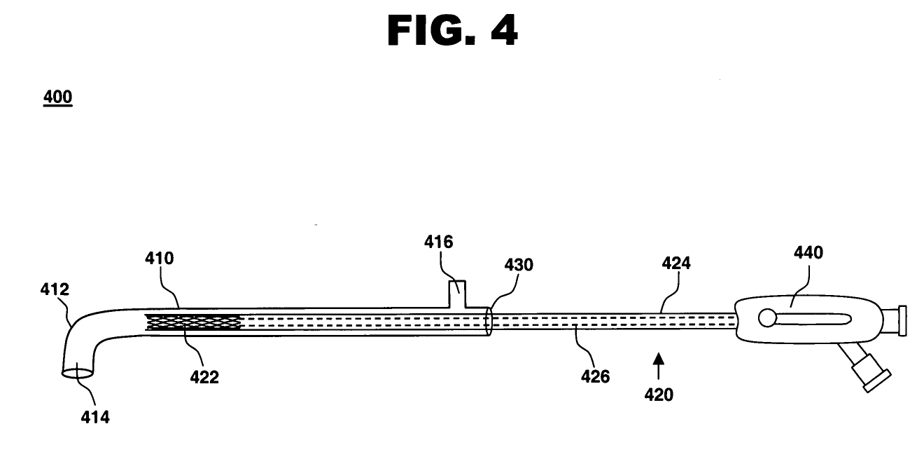

Another embodiment of the system, in accordance with the present invention, is illustrated in FIG. 4 at 400. System 400 comprises a guide catheter 410, a delivery catheter 420, a treatment device 422, and a stop interface 430.

Guide catheter 410, like guide catheter 110 described above, includes a shaped, flexible tip 412; a central lumen 414 that receives delivery catheter 420; and a flush port 416. In this embodiment, the treatment device disposed on delivery catheter 420 is self-expanding stent 422. Delivery catheter 420 has a sheath 426 that is withdrawn by sheath retractor 440 to allow expansion of self-expanding stent 422. As will be apparent to one skilled in the art, a system in accordance with the present invention may include both a sheath and a balloon, if desired, with the sheath protecting a therapeutic coating 426 on an expandable stent or with the balloon assisting in expansion of a self-expanding stent. Delivery catheter 420 may be directed through guide catheter 410 with or without the aid of a guidewire (not shown).

Stop interface 430 provides means for retaining a portion of delivery catheter 420 within the central lumen of guide catheter 410 while allowing positioning of a treatment device, in this embodiment stent 422, beyond the distal end of the guide catheter. In the present embodiment, stop interface 430 is the interface between guide catheter 410 and sheath 426, which may include a hemostatic valve to retain a portion of delivery catheter 424 within central lumen 412 while still allowing positioning of stent 422 beyond a distal end of guide catheter 410. Delivery catheter 420 or sheath 426 may include a stop 432 to prevent the treatment device from being damaged by coming into contact with the stop interface. Stop 432 may be, for example, a droplet of adhesive deposited onto the outer surface of delivery catheter 420. An end user receives system 400 with delivery catheter 420 already attached to guide catheter 410.

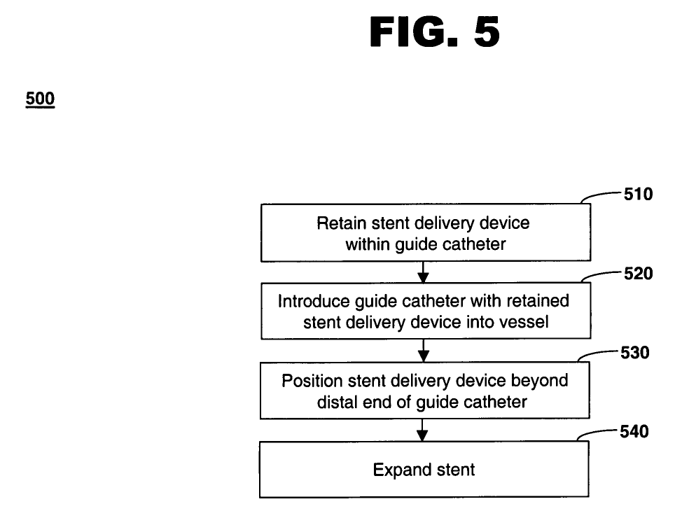

Another aspect of the present invention is a method for treating a vascular condition. FIG. 5 shows a flow diagram of one embodiment of the method in accordance with the present invention.

A stent delivery device is retained within a guide catheter, for example using a system such as one of those described above (Block 510). The guide catheter with retained stent delivery device is introduced into a vessel (Block 520). This may be accomplished by creating a percutaneous access site in a vessel to be treated or in a vessel that leads to the treatment site. The guide catheter and stent delivery device are then introduced through the percutaneous access site and advanced to a position adjacent to the desired treatment site. A distal portion of the guide catheter may comprise a shaped, flexible tip predisposed to bend at a predetermined angle, and this tip may direct the guide catheter into a vessel that branches off the vessel into which the guide catheter is initially introduced. For example, the flexible tip may direct the guide catheter into the internal iliac artery, which branches off the femoral artery, as shown in FIG. 2.

A stent carried on the stent delivery device is positioned at a location beyond a distal end of the guide catheter (Block 530). To accomplish this, the user may grasp a proximal portion of the stent delivery device that extends from the proximal end of the guide catheter and slide the stent delivery device within the guide catheter until the stent exits the distal end of the guide catheter.

The stent is then expanded (Block 540). Where the stent delivery system includes a balloon positioned within the stent, the balloon is inflated to expand the stent. Where the stent delivery system carries a self-expanding stent, a sheath positioned over the stent is retracted to allow expansion of the stent.

In an alternative embodiment, the method may include introducing a guidewire into the vessel to aid in delivering the guide catheter and stent delivery device to the desired treatment site within a vessel. For example, the guidewire may extend through a central lumen within the stent delivery device.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for treating a vascular condition, comprising:
a guide catheter including a central lumen;
a delivery catheter slidably received in the central lumen;
a treatment device disposed on the delivery catheter; and
a stop interface positioned adjacent to a proximal end of the guide catheter, wherein the stop interface retains a portion of the delivery catheter within the central lumen of the guide catheter and allows positioning of the treatment device beyond a distal end of the guide catheter.

2. The system of claim 1 wherein the treatment device is a stent.

3. The system of claim 2 wherein the delivery catheter includes a balloon used to expand the stent.

4. The system of claim 2 wherein the delivery catheter includes a sheath that retracts to allow expansion of the stent.

5. The system of claim 2 wherein the stent includes a therapeutic coating disposed on the stent.

6. The system of claim 5 wherein the therapeutic coating includes an agent selected from a group consisting of an antineoplastic agent, an antiproliferative agent, an antibiotic, an antithrombogenic agent, an anticoagulant, an antiplatelet agent, and an anti-inflammatory agent.

7. The system of claim 1 wherein a distal portion of the guide catheter comprises a shaped, flexible tip predisposed to bend at a predetermined angle.

8. The system of claim 1 wherein the length of the guide catheter is less than 50 centimeters.

9. The system of claim 1 wherein the stop interface is sized and shaped to slidably receive the delivery catheter.

10. The system of claim 1 wherein the stop interface is sized and shaped to slidably receive a guidewire.

11. A system for treating a vascular condition, comprising:
a guide catheter including a central lumen;
a delivery catheter slidably received in the central lumen;
a treatment device disposed on the delivery catheter; and
means for retaining a portion of the delivery catheter within the central lumen of the guide catheter while allowing positioning of the treatment device beyond a distal end of the guide catheter.

12. The system of claim 11 wherein the guide catheter includes means for directing a tip of the guide catheter into a side branch of a vessel.

13. The system of claim 11 wherein the delivery catheter includes means for expanding a stent.

14. The system of claim 11 wherein the treatment device includes means for delivering a therapeutic agent to the site of the vascular condition.

15. A method for treating a vascular condition, comprising:
retaining a stent delivery device within a guide catheter using a stop interface;
introducing the guide catheter with retained stent delivery device into a vessel;
positioning a stent carried on the stent delivery device at a location beyond a distal end of the guide catheter; and
expanding the stent.

16. The method of claim 15 further comprising:
introducing a guidewire into the vessel.

17. The method of claim 15 wherein a distal portion of the guide catheter comprises a shaped, flexible tip predisposed to bend at a predetermined angle, and wherein introducing the guide catheter with retained stent delivery device into a vessel comprises directing the catheter tip into a side branch of a vessel.

18. The method of claim 15 wherein positioning the stent at a location beyond a distal end of the guide catheter comprises grasping a proximal portion of the stent delivery device that extends from a proximal end of the guide catheter and sliding the stent delivery device within the guide catheter until the stent exits a distal end of the guide catheter.

19. The method of claim 15 wherein expanding the stent comprises inflating a balloon positioned within the stent.

20. The method of claim 15 wherein expanding the stent comprises retracting a sheath positioned over the stent.
